(19) United States
(12) Patent Application Publication
(10) Pub. No.: US 2006/0241687 A1
(43) Pub. Date: Oct. 26, 2006

(54) Septal occluder with pivot arms and articulating joints

(76) Inventors: Erik N. Glaser, Waltham, MA (US); David R. Widomski, New York, NY (US); Steven W. Opolski, Carlisle, MA (US); Sean T. Forde, Watertown, MA (US); John A. Wright JR., Lexington, MA (US); Kelli P. Church, Brookline, MA (US); Bill C. Smith, Woburn, MA (US); Carol A. Devellian, Topsfield, MA (US)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 11/377,501

(22) Filed: Mar. 16, 2006

Related U.S. Application Data
(60) Provisional application No. 60/662,502, filed on Mar. 16, 2005.

(51) Int. Cl. A61B 17/08

(52) U.S. Cl. 606/213

(57) ABSTRACT

The invention provides a septal occluder for closing a patent foramen ovale (PFO). The septal occluder includes a flexible central portion connecting two occlusion shells for the percutaneous transluminal treatment of an intracardiac defect. The septal occluder includes two occlusion shells connected by an elongate member capable of pivoting or articulating. The elongate member pivots or articulates at the juncture of the occlusion shell and the elongate member, according to one embodiment, or in the center of the elongate member, according to another embodiment. The elongate member can have one or more pivot points.

### RELATED APPLICATIONS

This application incorporates by reference, and claims priority to and the benefits of, U.S. Provisional Patent Application Ser. No. 60/662,502, filed on Mar. 16, 2005.

### TECHNICAL FIELD

This invention generally relates to devices and related methods for treating intracardiac defects. More particularly, the invention provides an intracardiac occluder with a plurality of arms pivotably joined to a flexible central body portion for the percutaneous closure of intracardiac defects.

### BACKGROUND

The human heart is divided into four compartments or chambers. The left and right atria are located in the upper portion of the heart and the left and right ventricles are located in the lower portion of the heart. The left and right atria are separated from each other by a muscular wall, the interatrial septum, while the ventricles are separated by the interventricular septum.

Either congenitally or by acquisition, abnormal openings, holes, or shunts can occur between the chambers of the heart or the great vessels, causing blood to flow therethrough. Such deformities are usually congenital and originate during fetal life when the heart forms from a folded tube into a four chambered, two unit system. The deformities result from the incomplete formation of the septum, or muscular wall, between the chambers of the heart and can cause significant problems. Ultimately, the deformities add strain on the heart, which can result in heart failure if they are not corrected.

One such deformity or defect, a patent foramen ovale, is a persistent, one-way, usually flap-like opening in the wall between the right atrium and left atrium of the heart. Since left atrial pressure is normally higher than right atrial pressure, the flap typically stays closed. Under certain conditions, however, right atrial pressure exceeds left atrial pressure, creating the possibility for abnormal right to left shunting that can allow blood clots to enter the systemic circulation. This is particularly worrisome to patients who are prone to forming venous thrombus, such as those with deep vein thrombosis or clotting abnormalities.

Nonsurgical (i.e., percutaneous) closure of patent foramen ovale, as well as similar intracardiac defects such as atrial septal defect, ventricular septal defect, and closure of the left atrial appendages, is possible using a variety of mechanical closure devices. These devices, which allow patients to avoid the potential side effects often associated with either surgical closure or standard anticoagulation therapies, typically consist of a metallic structural framework that is combined with either a synthetic or biological tissue scaffold material. The scaffold material encourages ingrowth and encapsulation of the device. The biological tissue can be derived from the tunica submucosa layer of the porcine small intestine.

One mechanical closure device is an intracardiac or septal occluder, including a proximal support structure supporting a proximal occlusion shell and a distal support structure supporting a distal occlusion shell. The distal support structure is coupled to the proximal support structure through a central body portion. Current devices typically include a rigid central body portion between the distal and proximal support structures. Such devices suffer, however, from several disadvantages, including reduced conformability.

### SUMMARY OF THE INVENTION

The present invention provides a device for occluding intracardiac defects. The device includes a pivot at the connection between the central body portion and the occlusion shells, a pivot within the central body portion, or both a pivot at the connection between the central body portion and the occlusion shells and a pivot within the central body portion, to enhance conformability, provide immediate closure to a defect, encourage faster tissue ingrowth, and create a lower septal occluder profile.

In one aspect, the invention is a septal occluder including a first body portion having a plurality of arms and a second body portion having a plurality of arms. Each of the arms has a free end and a pivot end and at least one of the arms has a pivot at its pivot end. The arm having the pivot moves independently of the other arms.

According to one embodiment, the pivot at the pivot end of the arm is a first loop connected to a second loop to form a pivotable joint. According to another embodiment, the pivot is a ball and socket joint. According to a further embodiment, the pivot is a pin and collar joint. And, according to another embodiment, the septal occluder further includes at least one centering coil spring.

In another aspect, the invention is a septal occluder including a first body portion having a plurality of arms, a second body portion having a plurality of arms, and a central body portion joining the first and second body portions. Each of the arms has a free end and a pivot end and at least one of the arms has a pivot at its pivot end. The arm having the pivot moves independently of the other arms. The central body portion further includes at least one pivot to pivotably join the first and second body portions.

According to one embodiment, the pivot is a first loop connected to a second loop to form a pivotable joint. According to another embodiment, the first loop and the second loop comprise a spring.

In another aspect, the invention is a septal occluder including a first body portion having a plurality of arms, a second body portion having a plurality of arms, and a central body portion joining the first and second body portions. At least a portion of the central body portion further includes a coiled spring to pivotably join the first and second body portions.

According to one embodiment, the septal occluder further includes a hollow cylinder surrounding at least a portion of the central body portion.

In another aspect, the invention is a septal occluder including a first body portion having a plurality of arms, a second body portion having a plurality of arms, and a central body portion joining the first and second body portions. At least a portion of the central body portion further includes a ball and loop joint to pivotably join the first and second body portions.

In another aspect, the invention is a septal occluder including a first body portion having a plurality of arms, a second body portion having a plurality of arms, and a central body portion joining the first and second body portions. At least a portion of the central body portion further includes a knob and cylinder joint to pivotably join the first and second body portions.

In another aspect, the invention is a septal occluder including a first body portion having a fixed end and a pivot end where the pivot end is a first socket and a second body portion having a fixed end and a pivot end where the pivot end is a second socket. The septal occluder also includes a central body portion including a first ball at a first end and a second ball at a second end. The first socket is pivotably joined with the first ball to form a first pivot and the second socket is pivotably joined with the second ball to form a second pivot. The first pivot and the second pivot are oriented in different planes.

In another aspect, the invention is a septal occluder including a first body portion having a fixed end and a pivot end where the pivot end is a first socket and a second body portion having a fixed end and a pivot end where the pivot end is a second ball. The septal occluder also includes a central body portion including a first ball at a first end and a second socket at a second end. The first socket is pivotably joined with the first ball to form a first pivot and the second socket is pivotably joined with the second ball to form a second pivot. The first pivot and the second pivot are oriented in different planes.

In another aspect, the invention is a septal occluder including a first body portion, a second body portion, and a central body portion. The central body portion includes a first elongate member and a second elongate member. The first elongate member flexibly joins the first body portion and the second body portion. The second elongate member extends circumferentially around at least a portion of the first elongate member.

According to one embodiment, the second elongate member is a stiffening member. According to a further embodiment, the second elongate member is a spring.

The foregoing and other objects, aspects, features, and advantages of the invention will become more apparent from the following description and from the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a cutaway view of the heart illustrating an intracardiac defect.

FIG. 2A is a perspective side view of a septal occluder according to one illustrative embodiment of the invention.

FIG. 2B is a perspective side view of a septal occluder according to another illustrative embodiment of the invention.

FIG. 2C is a perspective side view of a portion of a septal occluder according to another illustrative embodiment of the invention.

FIG. 3 is a side view of a septal occluder according to another illustrative embodiment of the invention.

FIG. 4A is a perspective side view of a septal occluder according to another illustrative embodiment of the invention.

FIG. 4B is a perspective side view of a portion of one embodiment of the illustrative septal occluder of FIG. 4A.

FIG. 4C is a side view of a portion of another embodiment of the illustrative septal occluder of FIG. 4A.

FIG. 5 is a side view of a septal occluder according to another illustrative embodiment of the invention.

FIG. 6 is a side view of a septal occluder according to another illustrative embodiment of the invention.

FIG. 7 is a perspective side view of a septal occluder according to another illustrative embodiment of the invention.

FIG. 8 is a side view of a septal occluder according to another illustrative embodiment of the invention.

FIGS. 9A and 9B are side views of the central body portion of a septal occluder according to other illustrative embodiments of the invention.

FIGS. 10A and 10B are perspective side views of a septal occluder according to other illustrative embodiments of the invention.

FIG. 11A is a perspective side cut-away view of the central body portion of a septal occluder according to another illustrative embodiment of the invention.

FIGS. 11B and 11C are top views of the collar of the central body portion of the septal occluder of FIG. 11A according to another illustrative embodiment of the invention.

FIG. 12 is a perspective side view of the central body portion of a septal occluder according to another illustrative embodiment of the invention.

FIG. 13 is a perspective side view of the central body portion of a septal occluder according to another illustrative embodiment of the invention.

### DETAILED DESCRIPTION OF THE INVENTION

The present invention provides an intracardiac occluder for the repair of intracardiac defects, such as, for example, a patent foramen ovale, an atrial septal defect, a ventricular septal defect, and left atrial appendages. The intracardiac occluder includes a central body portion comprising at least one pivot to pivotably join the two body portions of the septal occluder.

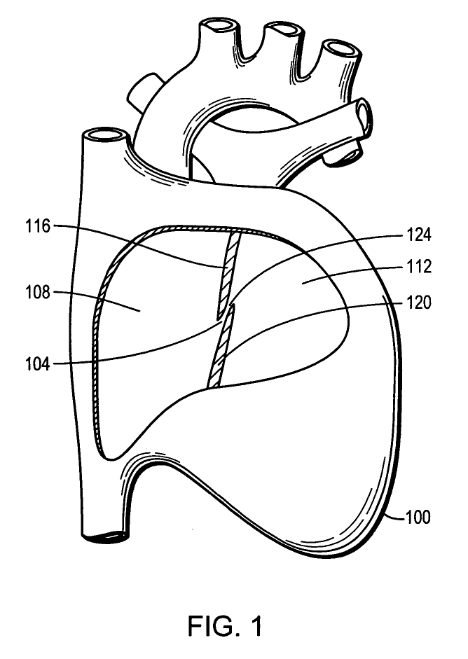

FIG. 1 depicts a cutaway view of a heart 100. The heart 100 includes a septum 104 that divides a right atrium 108 from a left atrium 112. The septum 104 includes a septum primum 120, a septum secundum 116, and an exemplary intracardiac defect, for example, a patent foramen ovale 124, which is to be corrected by the intracardiac occluder of the present invention, between the septum primum 120 and the septum secundum 116. Specifically, a patent foramen ovale 124 is shown as an opening through the septum 104. The patent foramen ovale 124 provides an undesirable fluid communication between the right atrium 108 and the left atrium 112. Under certain conditions, a large patent foramen ovale 124 in the septum 104 would allow for the shunting of blood from the right atrium 108 to the left atrium 112. If the patent foramen ovale 124 is not closed or obstructed in some manner, a patient is placed at high risk for an embolic stroke.

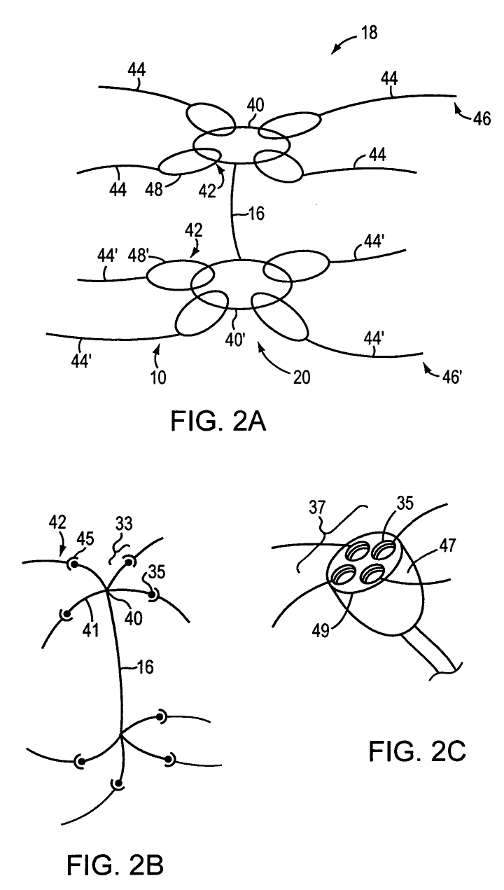

FIG. 2A is a perspective side view of a septal occluder 10 according to one illustrative embodiment of the invention. As shown, the septal occluder 10 includes a first body portion, for example, a proximal occlusion shell 18 (i.e., an occlusion shell that is closest to an operator of the septal occluder 10 (e.g., a physician)), a second body portion, for example, a distal occlusion shell 20, and a central body portion 16. The central body portion 16 joins the proximal occlusion shell 18 to the distal occlusion shell 20. According to one embodiment, each of the proximal occlusion shell 18 and the distal occlusion shell 20 contains four outwardly extending arms 44. Alternatively, either or both of the proximal and distal occlusion shells 18, 20 can contain two, three, five, six, or more arms 44 extending outwardly from the hub 40.

With continued reference to FIG. 2A, in one embodiment, each of the proximal occlusion shell 18 and the distal occlusion shell 20 includes a hub 40, from which a plurality of outwardly extending arms 44 are radially spaced. Each arm 44 includes a free end 46 and a pivot end 42. The pivot end 42 of the arm 44 is the end closest to the central body portion 16. The free end 46 is opposite to the pivot end 42. In one embodiment, each arm 44 connects to the hub 40 to form a pivot 48 at its pivot end 42. In another embodiment, at least one arm 44 connects to the hub 40 to form a pivot 48 at its pivot end 42. A pivot, as the term is used herein, is the point forming a fulcrum or the center on which something turns or oscillates. According to various embodiments, a pivot can be a loop joint, a hinge joint, a ball and socket joint, a pin and collar joint, a pin joint, a spring, or any other flexible structure capable of movement at one or more points known to one skilled in the art, or any other articulating connection or non-articulating joint deemed suitable by one skilled in the art.

According to one embodiment, the hub 40 forms a loop, as illustrated in FIG. 2A, a circle, an ellipse, or other shape forming a central opening to which each of the outwardly-extending arms 44 can be connected. According to one embodiment, each outwardly-extending arm 44 forms a complementary loop at its pivot end 42 that can interconnect with the loop of the hub 40 to form a pivot 48. According to one embodiment of the invention, each outwardly-extending arm 44 is individually connected to the loop of the hub 40 to form a pivot 48, allowing each individual arm 44 to pivot independently of the other arms 44. According to another embodiment of the invention, at least one arm 44 includes a pivot 48 at the pivot end 42. The arm 44 including the pivot 48 moves independently of the other arms 44 on the same occlusion shell 18 or 20. According to one embodiment of the invention, the pivot 48 can be placed at the hub 40 of the occlusion shell 18, 20. According to another embodiment, the pivot 48 can be placed at a distance along the arm 44 from the hub 40. The pivot 48 provides flexibility to the body portion 18, 20.

FIG. 2B is a perspective side view of a septal occluder 10 according to another illustrative embodiment of the invention. According to this alternative embodiment, the hub 40 of the septal occluder 10 includes multiple spokes 41, each spoke 41 extending from the central body portion 16. Each spoke 41 of the hub 40 terminates in a hemi-pivot member 35 that forms a pivot 33 with a complementary hemi-pivot member 45 on the pivot end 42 of the outwardly-extending arm 44. The pivot 33 can include, for example, a ball and socket joint, a pin and collar joint, a pin joint, or other joint known to one skilled in the art. According to the embodiment illustrated in FIG. 2B, each spoke 41 of the hub 40 terminates in a ball 35 and each outwardly-extending arm 44 terminates in a socket 45 to form a ball and socket pivot 33. According to an alternative embodiment, each spoke 41 terminates in a socket 45 and each outwardly-extending arm 44 terminates in a ball 35 to form a ball and socket pivot 33. In one embodiment, each outwardly-extending arm 44 is connected to the hub 40 by a pivot 33, allowing each arm 44 to pivot independently of the other arms 44. In another embodiment, at least one outwardly-extending arm 44 is connected to the hub 40 by a pivot 33, allowing the arm 44 to pivot independently of the other arms 44.

FIG. 2C is a perspective side view of a portion of a septal occluder 10 according to another illustrative embodiment of the invention. According to this illustrative embodiment, the hub 40 of the septal occluder 10 forms a hemi-pivot member that forms a single chamber or socket 47 with a complementary hemi-pivot member of the pivot end 42 of an outwardly-extending arm 44. In one embodiment, each outwardly-extending arm 44 terminates in a ball 35 at its pivot end 42. In another embodiment, at least one outwardly-extending arm 44 terminates in a ball 35 at its pivot end 42. The single chamber or socket 47 of the hub 40 is capable of accommodating one or multiple balls 35, for example, but not limited to, two, three, four, five, six, or more balls 35, to form a pivot 37. In one embodiment of the invention, each outwardly-extending arm 44 terminates in a ball 35 at its pivot end 42 connected to the single chamber or socket 47 of the hub 40 forming a ball and socket pivot 37. In another embodiment of the invention, at least one outwardly-extending arm 44 terminates in a ball 35 at its pivot end 42 connected to the single chamber or socket 47 of the hub 40 forming a pivot. According to alternative embodiments, the pivot 37 is a ball and socket joint, a pin and collar joint, a pin joint, or other joint known to one skilled in the art. According to one embodiment of the invention, each outwardly-extending arm 44 is able to pivot independently of the other arms 44. According to another embodiment, at least one outwardly-extending arm 44 is able to pivot independently of the other arms 44.

With continued reference to FIG. 2C, according to one embodiment, the single chamber or socket 47 contains a single opening 49 capable of accommodating one or multiple balls 35 and their outwardly-extending arms 44. According to the illustrative embodiment depicted in FIG. 2C, the single chamber or socket 47 is capable of accommodating four balls 35 within the single chamber or socket 47. According to another embodiment, the single chamber or socket 47 contains more than one (not shown) opening 49, for example, one opening 49 for each ball 35.

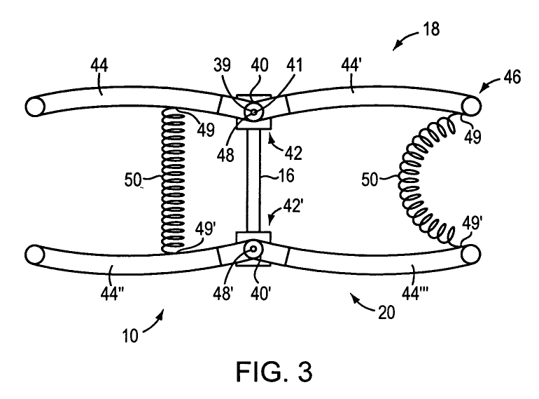

FIG. 3 is a side view of a septal occluder 10 according to another illustrative embodiment of the invention. As shown, the septal occluder 10 includes a proximal occlusion shell 18, a distal occlusion shell 20, and a central body portion 16. According to one embodiment, the central body portion 16 includes an elongate member having a first end and a second end. The first end of the elongate member of the central body portion 16 is pivotably joined to the hub 40 of one of the occlusion shell 18, 20. In another embodiment, the first end of the elongate member is pivotably joined to the proximal occlusion shell and the second end of the elongate member is pivotably joined to the distal occlusion shell 20.

With continued reference to FIG. 3, according to one embodiment, each of the proximal occlusion shell 18 and the distal occlusion shell 20 includes a hub 40, from which a plurality of outwardly extending arms 44 are radially spaced. Each arm 44 includes a free end 46 and a pivot end 42. According to one embodiment, the pivot end 42 of a first arm 44 terminates in a pin 41 and the pivot end 42 of an opposite arm 44′ terminates in a socket 39 for receiving the pin 41. Alternatively, according to another embodiment, the pivot end 42 of both the first arm 44 and the second arm 44′ terminate a socket 39 for receiving a pin 41. Additionally, according to alternative embodiments, the first end and/or the second end of the central body portion 16 can terminate in a pin 41 or a socket 39 for receiving a pin 41. Accordingly, the first arm 44 and the opposite arm 44′ are pivotably joined at the hub 40 and either arm can pivot independently of the other arm. The pivot can be modified to either allow flexibility and conformability of the arms 44 or it can be modified to inhibit complete freedom of movement, for example, by adding a mechanical stop near the pivot that would limit the motion of the arm.

With continued reference to FIG. 3, according to one embodiment, the septal occluder 10 can include at least one centering coil spring 50 attached to, for example, an attachment point 49 on an arm 44 of the proximal occlusion shell 18 and a second attachment point 49′ on an arm 44″ of the distal occlusion shell 20, to maintain the arms 44, 44″ of the proximal occlusion shell 18 and the distal occlusion shell 20 under tension. The centering coil spring 50 can be formed of an elastic material, such as a metal alloy, plastic, or rubber configured in a shape designed to maintain the arms 44, 44″ a maximum or minimum separation distance. The attachment point 49 may be positioned on the arm 44 at any position from the free end 46 to the pivot end 42.

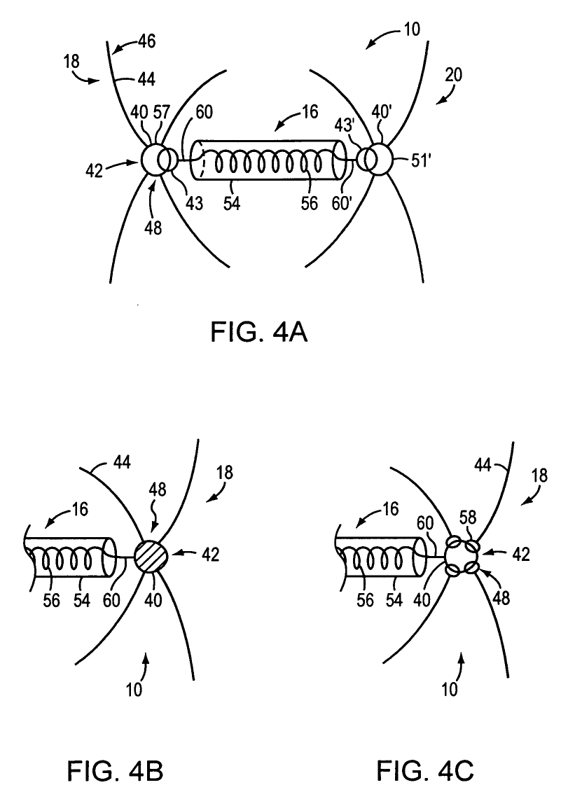

FIG. 4A is perspective side view of a septal occluder 10 according to another illustrative embodiment of the invention. FIGS. 4B and 4C are side views of a portion of the alternative embodiments of the illustrative septal occluder 10 of FIG. 4A. Referring to FIG. 4A, the septal occluder 10 includes a proximal occlusion shell 18, a distal occlusion shell 20, and a central body portion 16. In one embodiment, each of the proximal occlusion shell 18 and the distal occlusion shell 20 includes a hub 40, from which a plurality of outwardly extending arms 44 are radially spaced. The hub 40 according to this illustrative embodiment can include any of the hub configurations described herein with respect to alternative illustrative embodiments of the hub of the invention or a combination of the alternative hub elements described therewith.

With continued reference to FIG. 4A, according to one embodiment, the central body portion 16 includes an elongate member, for example, a cylinder 54 enclosing a longitudinally oriented coiled spring 56. The coiled spring 56 can be formed of an elastic material, such as a metal alloy, plastic, or rubber configured in a shape designed to maintain the proximal and distal occlusion shells 18, 20 a maximum or minimum separation distance. According to one embodiment, a first loop 43, 43′ is formed on each end of the coiled spring 56, capable of forming a pivot 48 when joined with a second loop 51, 51′ on the hub 40, 40′ of the occlusion shell 18, 20. According to another embodiment, a ball (not shown) is formed on each end of the coiled spring 56, capable of forming a pivot 48 when joined with a socket (not shown) on the hub 40 of the occlusion shell 18, 20.

Referring to FIG. 4B, according to another embodiment, the coiled spring 56 of the central body portion 16 is connected to the proximal occlusion shell 18 by a pin 60 and to the distal occlusion shell 20 by a second pin 60′ (not shown). Each pin 60 is connected to the coiled spring 56 on one end and to the hub 40 on the other end. According to one embodiment, each pin 60 is attached to the coiled spring 56 and/or the hub 40, or pivot, directly, for example, by a laser weld, by a wire, or by other connectors deemed suitable by one skilled in the art. According to another embodiment, each pin 60 is pivotably joined to the coiled spring 56 and/or the hub 40, to form a pivot 48, through, for example, a loop connection, a ball and socket joint, a pin and collar joint, a pin joint, a spring or other flexible structure known to one skilled in the art.

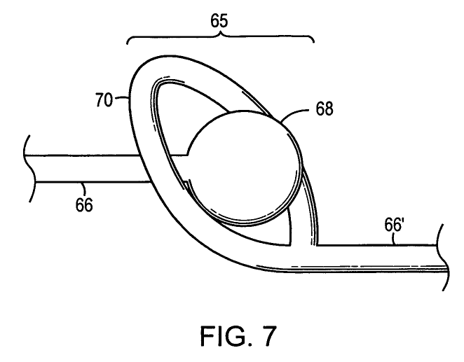

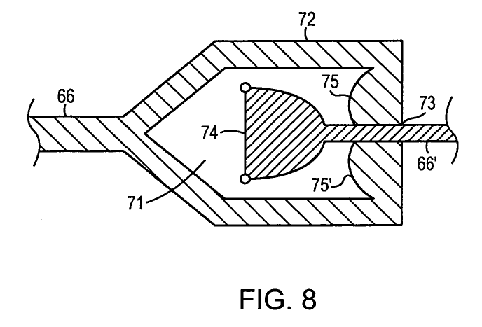

Referring still to FIG. 4B, according to one embodiment of the invention, the pivot end 42 of the outwardly extending arms 44 of the occlusion shell 18, 20 can attach to the pin 60 of the central body portion 16 at the hub 40 directly, for example, by a laser weld, by a wire (not shown), or by other connectors deemed suitable by one skilled in the art. According to another embodiment of the invention, depicted in FIG. 4C, the pivot end 42 of the outwardly extending arms 44 can attach to the hub 40 through wire loops 58, allowing each of the arms 44 to move independent of one another. According to another embodiment of the invention, the pivot end 42 of the outwardly extending arms 44 can form a ball (not shown) that can attach to a socket or chamber (not shown) on the hub 40, described below with respect to FIGS. 7 and 8, allowing the arms 44 to move independent of one another.

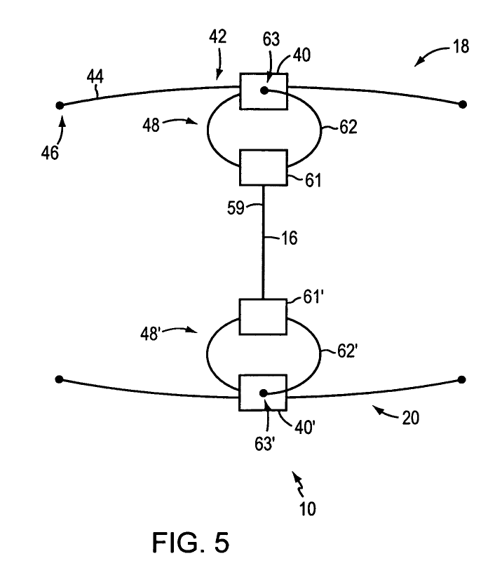

FIG. 5 is a side view of a septal occluder 10 according to another illustrative embodiment of the invention. As shown, the exemplary septal occluder 10 includes a proximal occlusion shell 18, a distal occlusion shell 20, and a central body portion 16. According to one embodiment, each of the proximal occlusion shell 18 and the distal occlusion shell 20 includes a hub 40, from which a plurality of outwardly extending arms 44 are radially spaced. Each outwardly extending arm 44 of an occlusion shell 18, 20 includes a free end 46 and a pivot end 42. In one embodiment, each arm 44 connects to the hub 40 to form a pivot 48 at its pivot end 42. In another embodiment, at least one arm 44 connects to the hub 40 to form a pivot 48 at its pivot end 42. The arm 44 including the pivot 48 moves independently of the other arms 44 on the same occlusion shell 18, 20.

With continued reference to FIG. 5, the central body portion 16 of the septal occluder 10 includes a rod 59 having a first end and a second end, containing an attachment point 61, 61′ at each end. The attachment points 61, 61′ can be, for example, in the form of a loop, a ball with a lumen extending from one side of the rod to another side, a block with a lumen extending from one side of the rod to another side, or another configuration adapted to accept and contain a wire that passes through the lumen 63, 63′. The hub 40 of the proximal occlusion shell 18 contains a lumen 63 that extends from one side of the hub 40 to another side of the hub 40, and is connected to the attachment point 61 of the central body portion 16 by, for example, a loop 62 extending from the attachment point 61 of the central body portion 16 and through the lumen 63 of the hub 40. The hub 40′ of the distal occlusion shell 20 also contains a lumen 63′ that extends from one side of the hub 40′ to another side of the hub 40′, and is connected to the attachment point 61′ of the central body portion 16 by, for example, a second loop 62′ extending from the attachment point 61′ of the central body portion 16 and through the lumen 63′ of the hub 40′. The loops 62, 62′ can be formed of a wire formed of metal, a metal alloy, plastic, or other suitable materials known to those skilled in the art. Each of the proximal and distal occlusion shells 18, 20 can pivot or rotate independent of each other or the central body portion 16.

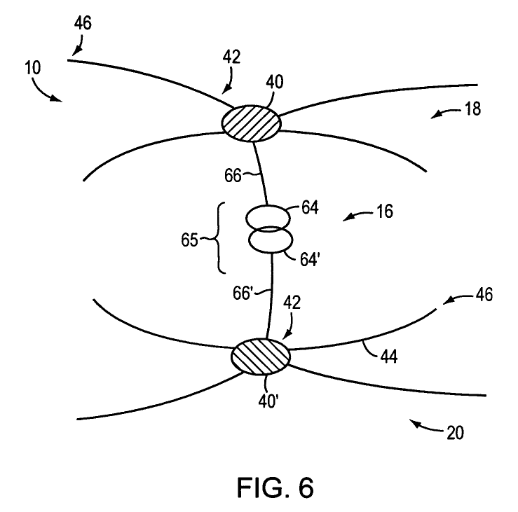

FIG. 6 is a side view of a septal occluder 10 according to another illustrative embodiment of the invention. As shown, the septal occluder 10 includes a proximal occlusion shell 18, a distal occlusion shell 20, and a central body portion 16. The plurality of outwardly extending arms 44 of each of the proximal and distal occlusion shells 18, 20 attach to the hub 40 at their pivot ends 42. The central body portion 16 includes two pins 66, 66′ each having a first end and a second end. In one embodiment, the first pin 66 is attached, on its first end, to the hub 40 of the proximal occlusion shell 18 by, for example, a laser weld, a wire (not shown), or other connectors deemed suitable by one skilled in the art, and contains a first loop 64 on its second end. In another embodiment, the second pin 66′ is attached to the hub 40′ of the distal occlusion shell 20 on its first end and contains its second loop 64′ on a second end. The first loop 64 and the second loop 64′ interconnect to form a pivot 65 at a point between the proximal hub 40 and the distal hub 40′, such that the two pins 66, 66′ and their attached occlusion shells 18, 20 are pivotably joined about the pivot 65. According to one embodiment, the pivot 65 is a loop connection between a first loop 64 and a second loop 64′. According to an alternative embodiment, the central body portion 16 can include more than one pivot 65, for example, the central body portion 16 can include two, three, four, or more than four pivots. Additionally, according to another embodiment, the pivot 65 of the central body portion 16 can include a connection of more than two loops, for example, a loop connection between a first loop 64, a second loop 64′, and also a third loop (not shown), a fourth loop (not shown) or more loops, providing additional flexibility to the central body portion 16. According to one embodiment, the pins 66, 66′ and the loops 64, 64′ are formed of a wire of metal, a metal alloy, plastic, rubber, or other suitable rigid or semi-rigid material. According to alternative embodiments, the pins 66, 66′ are attached to the hubs 40, 40′ by a pivot 65, such as an interconnecting loop joint (not shown), a ball and socket joint (not shown), a pin and collar joint (not shown), or another joint configuration described with respect to an alternative embodiment of the invention herein, or known by one of skill in the art.

FIG. 7 is a perspective side view of a portion of the central body portion 16 of a septal occluder 10 according to another illustrative embodiment of the invention. The septal occluder (not shown) includes a proximal occlusion shell (not shown), a distal occlusion shell (not shown), and a central body portion 16. The plurality of outwardly extending arms (not shown) of the proximal and distal occlusion shells (not shown) attach to the hub (not shown) at their pivot ends (not shown).

With continued reference to FIG. 7, in one embodiment, the pin 66 of the exemplary central body portion 16 is attached to the proximal occlusion shell (not shown) at its first end and terminates in a ball 68 at its second end. The second pin 66′ is attached to the distal occlusion shell (not shown) at its first end and terminates in a socket 70 at its second end. According to one embodiment, the socket 70 is in the form of a wire loop. According to another embodiment, the socket 70 is in the form of a cup. It is contemplated that the location of the ball 68 and socket 70 can be reversed, such that the ball 68 is connected to the distal occlusion shell and the socket 70 is connected to the proximal occlusion shell. According to this embodiment, the ball 68 is held within the socket 70 forming a pivot 65 at a point between the proximal hub and the distal hub such that the two pins 66, 66′ and their attached occlusion shells are pivotably joined about the pivot 65. According to another embodiment, the pins 66, 66′ are attached to the proximal and distal hubs by a pivot (not shown), such as an loop connection (not shown), a ball and socket joint (not shown), a pin and collar joint (not shown), or another joint configuration described with respect to an alternative embodiment of the invention herein, or known by one of skill in the art.

FIG. 8 is a cut-away side view of a portion of the central body portion 16 of the septal occluder 10 of FIG. 6 according to another illustrative embodiment of the invention. According to this embodiment of the invention, a first elongate member of the central body portion 16, for example, a pin 66, includes a first end and a second end. The pin 66 is attached to the proximal occlusion shell (not shown) at a first end and contains a collar 72 at a second end. The collar 72, according to the illustrative embodiment, is a hollow cylinder including an interior lumen 71. According to another embodiment, the hollow cylinder 72 further includes a first ridge 75 and a second ridge 75′. The collar 72 also includes an opening 73 extending from the interior lumen 71 to the exterior surface opposite to the pin 66 and occlusion shell (not shown).

With continued reference to FIG. 8, the second elongate member of the central body portion 16, for example, a pin 66′, also includes a first end and a second end. The pin 66′ is attached to the distal occlusion shell (not shown) at a first end and includes a knob 74 at a second end. The knob 74, according to one embodiment, is in the form of a hemisphere with the narrower portion of the hemisphere closest to the attached second pin 66′. It is contemplated that the knob 74 can be formed in additional shapes, such as a ball, a pyramid, a polyhedron, or another shape having a wide portion furthest away from the second pin 66′ and a narrowing toward the second pin 66.

With continued reference to FIG. 8, the second pin 66′ and the knob 74 is inserted through the opening 73 of the collar 72. In this configuration, the knob 74 is contained within the interior lumen 71 of the collar 72. According to another embodiment, the collar 72 includes a ridge 75 on the interior luminal surface surrounding the opening 73 that retains the knob 74 within the interior lumen 71 of the collar 72 and prevents the knob 74 from pivoting beyond a fixed predetermined angle. It is contemplated that the location of the knob 74 and collar 72 can be reversed, such that the knob 74 is connected by the first pin 66 to the proximal occlusion shell and the collar 72 is connected by the second pin 66′ to the distal occlusion shell. According to another embodiment, the pins 66, 66′ are attached to the proximal and distal occlusion shells 18, 20 by a pivot 65, such as a loop connection (not shown), a ball and socket joint (not shown), a pin and collar joint (not shown), or another joint configuration described with respect to an alternative embodiment of the invention herein, or known by one of skill in the art.

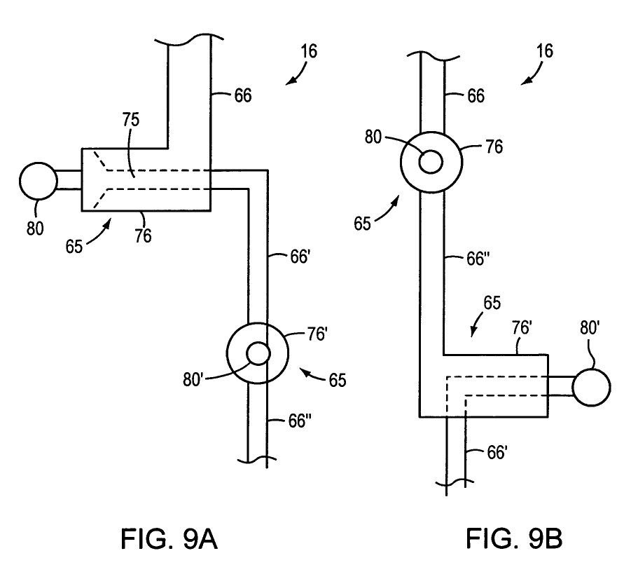

FIGS. 9A and 9B are side views of a portion of the central body portion 16 of a septal occluder 10 according to another illustrative embodiment of the invention. According to this illustrative embodiment, the central body portion 16 comprises multiple pivots 65, formed by balls (80 generally) joined to sockets (76 generally). Further, each pivot 65 allows the connecting pins 66 to rotate 360 degrees within a single plane. When two or more pivots 65 combine, each within an alternate plane, for example, in two planes oriented at 90 degrees relative to one another, the proximal occlusion shell (not shown) and the distal occlusion shell (not shown) articulate relative to one another through 360 degrees of rotation in any plane, allowing the central body portion 16 to adapt to the geometry of the intracardiac defect and provide a complete closure of the intracardiac defect.

Referring to the exemplary embodiment of the invention illustrated in FIG. 9A, the first pin 66 of the central body portion 16 is attached at one end to the proximal occlusion shell (not shown) and contains a first socket 76 at the other end. The second pin 66′ is attached at one end to the distal occlusion shell (not shown) and contains a second socket 76′ at the other end. Each socket 76, 76′ contains a lumen 75 through which a third pin 66″ can be inserted. The third pin 66″ terminates in a first ball 80 on its first end. The first ball 80 has a radius larger than the opening of the first socket 76, holding the third pin 66″ within the first socket 76 forming a pivot 65. The third pin 66″ also terminates in a second ball 80′ on its second end. The second ball 80′ has a radius larger than the opening of the second socket 76′, holding the third pin 66″ within the second socket 76′ forming a second pivot 65′. According to an alternative embodiment, the third pin 66″ terminates in another anchoring mechanism such as a ball (not shown) on its first end and second end to hold the third pin 66″ within the first socket 76 and second socket 76′.

Referring to FIG. 9B, in an alternative embodiment, either or both the first pin 66 and/or the second pin 66′ terminate in a ball 80. According to this embodiment, either or both ends of the third pin 66″ contain a socket 76, such that each pivot 65 between two pins 66 contains one ball 80 joined to one socket 76. Additionally, it is contemplated that the central body portion 16 contains one, two, three, four, or more pivots 65. Additionally, the pivot 65 can be at an angle greater than 90 degrees, for example, an angle in the range of 90 to 180 degrees.

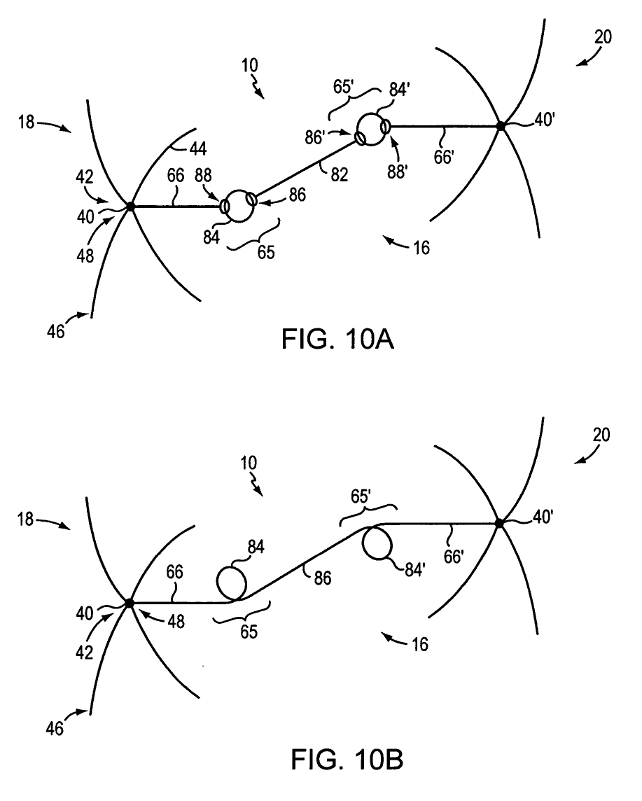

FIGS. 10A and 10B are perspective side views of a septal occluder 10 according to another illustrative embodiment of the invention. As shown in FIG. 10A, the septal occluder 10 includes a proximal occlusion shell 18, a distal occlusion shell 20, and a central body portion 16. The plurality of outwardly extending arms 44 of the proximal and distal occlusion shells 18, 20 attach to the hub 40 at their pivot ends 48. The central body portion 16 includes two pivots 65, 65′. According to one embodiment, the first pin 66 of the central body portion 16 is attached at its first end to the proximal occlusion shell (not shown) and rigidly connected to the first ring 84 at its second end, the second pin 66′ of the central body portion 16 is attached at its first end to the distal occlusion shell (not shown) and rigidly connected to the second ring 84′ at its second end. The two rings 84, 84′ are connected by an elongate member, for example, a central rod 82. According to this embodiment, the central rod 82 contains a first loop 86 and a second loop 86′, one at each end of the central rod 82. The first loop 86 is interconnected with the first ring 84 forming a pivot 65 and the second loop 86′ is interconnected with the second ring 84′ forming a pivot 65′. According to this embodiment, the central rod 82 pivots about both the first ring 84 and the second ring 84′, providing two pivots within the central body portion 16.

With continued reference to FIG. 10A, according to an alternative embodiment of the invention, the first pin 66 of the central body portion 16 is attached at its first end to the proximal occlusion shell (not shown) and includes a first loop 88 at its second end. The first loop 88 is interconnected with the first ring 84 forming a first pivot 65. Additionally, the second pin 66′ of the central body portion 16 is attached at its first end to the distal occlusion shell (not shown) and includes a second loop 88′ at its second end. The second loop 88′ is interconnected with the second ring 84′ forming a second pivot 65′. According to an alternative embodiment, the central rod 82 rigidly connects to the first ring 84 and the second ring 84′. It is also contemplated that the second end of the first pin 66, the second end of the second pin 66′, and each end of the central rod 82 can contain a loop 86, 86′, 88, 88′, each interconnected with either the first ring 84 or the second ring 84′ to form a pivot 65. Other combinations of loops and rings are contemplated, such that the central body portion 16 contains at least two pivots.

According to the exemplary embodiment of the invention in FIG. 10B, a single piece of wire forms the two rings 84, 84′, the rod 86, and the pins 66, 66′ of the central body portion 16. According to this embodiment, the single wire is shaped such that the two rings 84, 84′ are each a single coil spring that exert a restorative tension to maintain the proximal and distal occlusion shells 18, 20 a distance apart, but that allow the central body portion 16 to extend or contract and to pivot about a central axis to allow the proximal and distal occlusion shells 18, 20 maximum flexibility.

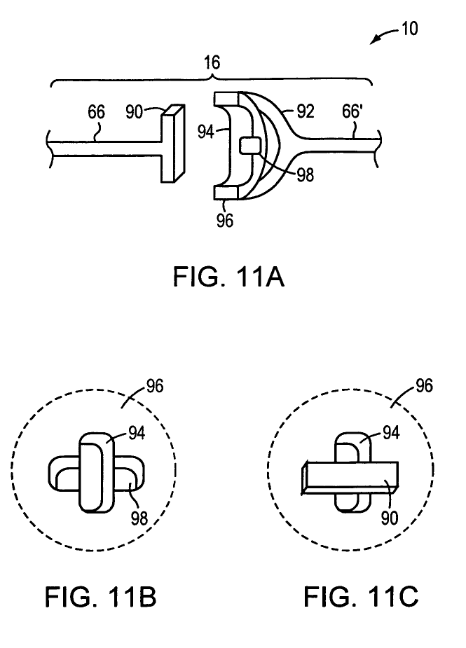

FIGS. 11A, 11B and 11C are side views of a portion of the central body portion 16 of a septal occluder 10 according to another illustrative embodiment of the invention. FIG. 11A is a cut-away side view of a portion of the central body portion 16 of the septal occluder 10. FIGS. 11B and 11C are top views of the collar 96 of the central body portion 16 of the septal occluder 10 of FIG. 11A according to another illustrative embodiment of the invention. According to one embodiment of the invention, a first pin 66 attaches at its first end to the proximal occlusion shell (not shown) and contains a block 90 at its second end. A second pin 66′ attaches at its first end to the distal occlusion shell (not shown) and includes a collar 96 comprising a lumen 94 and a ridge 98 at its second end. The location of the block 90 and collar 96 can be reversed, such that the second end of the second pin 66′ contains the block 90 and the second end of the first pin 66 contains the collar 96. According to one embodiment of the invention, the block 90 inserts into the lumen 94 of the collar 96, twisted ninety degrees in either direction, and partially retracted, into the ridge 98, such that the block 90 and collar 96 interlock (as depicted in FIG. 11C) forming a pivot.

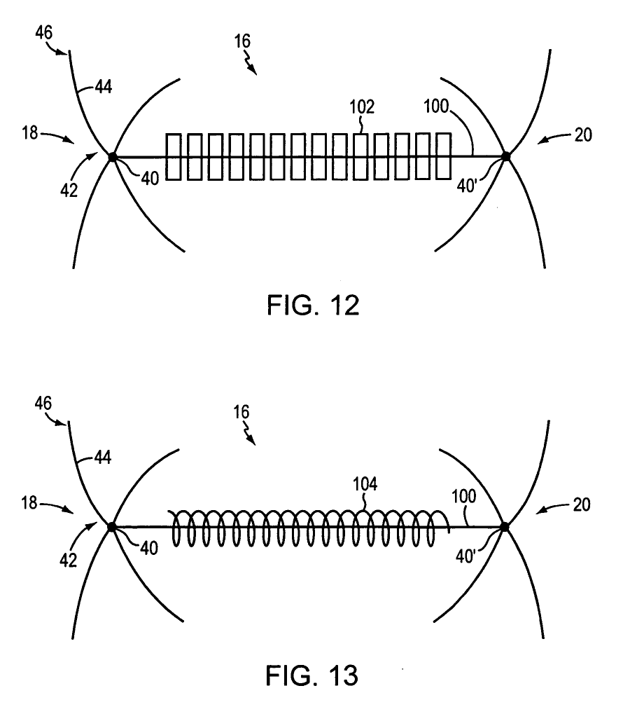

FIG. 12 is a perspective side view of the septal occluder 10 according to another illustrative embodiment of the invention. According to the embodiment of the invention depicted in FIG. 12, the central body portion 16 of the septal occluder 10 comprises a first elongate member 100 and a second elongate member 102. According to one embodiment, the first elongate member 100 connects at a first end to the hub 40 of the proximal occlusion shell 18 and at a second end to the hub 40′ of the distal occlusion shell 20. A second elongate member 102, for example, a stiffening member, is disposed circumferentially around the first elongate member 100 and extends along at least a portion of the length of the first elongate member 100.

With continued reference to the embodiment of the invention illustrated in FIG. 12, according to one embodiment, the first elongate member 100 has a greater flexibility than the second elongate member 102, for example, due to the material selected to form the elongate member or the method of forming the elongate member. The second elongate member 102, for example, a stiffening member, is formed of an elastic material, such as a metal alloy, plastic, or rubber configured in a shape designed to maintain the proximal and distal occlusion shells 18, 20 a maximum or minimum separation distance. The second elongate member 102 imparts additional rigidity to the central body portion 16, for example, along that portion of the central body portion 16 that spans the intracardiac defect, while allowing the first elongate member 100 to move with additional flexibility, for example, along those portions of the central body portion 16 closest to the proximal and distal occlusion shells 18, 20, allowing the occlusion shells 18, 20 a greater relative freedom of movement at the junction with the central body portion 16 while retaining a greater relative rigidity over the portion of the central body portion 16 contacting the intracardiac defect.

FIG. 13 is a perspective side view of the septal occluder 10 according to another illustrative embodiment of the invention. According to the embodiment of the invention depicted in FIG. 13, the central body portion 16 comprises a first elongate member 100 and a second elongate member 104. According to one embodiment, the first elongate member 100 connects at a first end to the hub 40 of the proximal occlusion shell 18 and at a second end to the hub 40′ of the distal occlusion shell 20. A second elongate member 104, for example, a braided cable, a helical spring, a spiral spring, or another stiffening member, is disposed circumferentially around the first elongate member 100 and extends along at least a portion of the length of the first elongate member 100. The second elongate member 104 is formed of an elastic material, such as a metal alloy, plastic, or rubber configured in a shape designed to maintain the proximal and distal occlusion shells 18, 20 a maximum or minimum separation distance.

With continued reference to FIG. 13, according to one embodiment, the first elongate member 100 is flexibly joined to both the proximal occlusion shell 18 and the distal occlusion shell 20, and the second elongate member 104 extends circumferentially around and along at least a portion of the first elongate member 100, but is not connected to either of the occlusion shells 18, 20. According to another embodiment, both the first elongate member 100 and the second elongate member 104 are flexibly joined to each of the proximal occlusion shell 18 and the distal occlusion shell 20. The combination of the first elongate member 100 and the second elongate member 104 allow the central body portion 16 to stretch to a given length or with a given amount of resistive stretching force to make the central body portion 16 more conformable and to add flexibility.

Variations, modifications, and other implementations of what is described herein will occur to those of ordinary skill in the art without departing from the spirit and the scope of the invention as claimed. Accordingly, the invention is to be defined not by the preceding illustrative description, but instead by the spirit and scope of the following claims.

1. A septal occluder, comprising:
a first portion comprising a plurality of arms; and
a second portion comprising a plurality of arms;
wherein each of said arms comprises a free end and a pivot end, at least one of the arms comprises a pivot at said pivot end, and said arm comprising a pivot moves independently of the other arms.

2. The septal occluder of claim 1 wherein the pivot comprises a first loop connected to a second loop.

3. The septal occluder of claim 1 wherein the pivot comprises a ball and loop joint.

4. The septal occluder of claim 1 wherein the pivot comprises a ball and socket joint.

5. The septal occluder of claim 1 wherein the pivot comprises a pin and collar joint.

6. The septal occluder of claim 1 further comprising at least one centering coil spring.

7. A septal occluder, comprising:
a first body portion comprising a plurality of arms;
a second body portion comprising a plurality of arms;
a central body portion joining said first body portion and said second body portion and comprising at least one pivot, wherein said central body portion pivotably joins said first and said second body portions; and
wherein each of said arms comprises a free end and a pivot end, at least one of the arms comprises a pivot at said pivot end, and said arm comprising a pivot moves independently of the other arms.

8. The septal occluder of claim 7 wherein the central body portion pivot comprises at least a first loop connected to a second loop to form a pivotable joint.

9. The septal occluder of claim 8 wherein the first loop and the second loop further comprise a spring.

10. The septal occluder of claim 7 wherein the central body portion pivot comprises a ball and socket joint.

11. A septal occluder, comprising:
a first body portion comprising a plurality of arms;
a second body portion comprising a plurality of arms; and
a central body portion joining said first body portion and said second body portion wherein at least a portion of said central body portion comprises a coiled spring and said central body portion pivotably joins said first and said second body portions.

12. The septal occluder of claim 11 further comprising a hollow cylinder surrounding at least a portion of the central body portion.

13. A septal occluder, comprising:
a first body portion comprising a plurality of arms;
a second body portion comprising a plurality of arms; and
a central body portion joining said first body portion and said second body portion and comprising a ball and loop joint, wherein said central body portion pivotably joins said first and said second body portions.

14. A septal occluder, comprising:
a first body portion comprising a plurality of arms;
a second body portion comprising a plurality of arms; and
a central body portion joining said first body portion and said second body portion and comprising a knob and cylinder joint, wherein said cylinder comprises a lumen and a ridge and the knob contacts the ridge in the lumen of the cylinder, and said central body portion pivotably joins said first and said second body portions.

15. A septal occluder comprising:
a first body portion comprising a fixed end and a pivot end, said pivot end comprising a first socket;
a second body portion comprising a fixed end and a pivot end, said pivot end comprising a second socket; and
a central body portion comprising a first ball at a first end and a second ball at a second end, wherein said first socket is pivotably joined with said first ball to form a first pivot, and said second socket is pivotably joined with said second ball to form a second pivot; and wherein said first pivot and said second pivot are oriented in different planes.

16. A septal occluder, comprising:
a first body portion;
a second body portion; and
a central body portion comprising a first elongate and a second elongate member, said first elongate member flexibly joining said first body portion and said second body portion, and said second elongate member extending circumferentially around at least a portion of the first elongate member.

17. The septal occluder of claim 16 wherein said second elongate member further comprises a stiffening member.

18. The septal occluder of claim 17 wherein said second elongate member further comprises a spring.
